(19) United States
(12) Patent Application Publication
(10) Pub. No.: US 2004/0167061 A1
(43) Pub. Date: Aug. 26, 2004

(54) Substantially homogeneous bio-affecting material having a pre-determined ratio of bioaffecting component to cell targeting component, the method for making such a material and the method of its use

(76) Inventor: W. Page Faulk, Indianapolis, IN (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/477,999

(22) PCT Filed: May 15, 2002
(86) PCT No.: PCT/US02/11891

(51) Int. Cl. C07K 14/79; A61K 38/40

(52) U.S. Cl. 514/8; 530/395

(57) ABSTRACT

A homogeneous conjugate for targeting and treating diseased cells wherein the conjugate has a predetermined ratio of drug molecules to protein molecules that preferentially bind to such cells and a method for making such a conjugate. The method of making the conjugate comprises adding drug molecules to linker molecules in a manner that effectively results in one molecule of drug for each molecule of linker followed by the addition of the drug-linker combination to protein molecules in order to achieve the predetermined ratio of drug molecules to protein molecules.

### FIELD OF THE INVENTION

This invention relates generally to the field of bio-affecting materials and more specifically to substantially homogeneous protein-drug conjugates, the method of their making and the method of their use.

### BACKGROUND OF THE INVENTION

Two of the most devastating problems in cancer treatment are drug-toxicity, which debilitates patients, and drug-resistance, which is normally countered with even higher drug dosages and thus amplifies the problem of drug-toxicity, often resulting in death. One way to solve the problem of drug-toxicity is to deliver drugs so they are targeted only to cancer cells. Many researchers are working to develop antibodies to deliver drugs, and this approach holds promise, but antibodies are not without problems. For example, antibodies often bind to normal tissues, and they also can damage blood vessels (e.g., vascular leak syndrome) and cause dangerous allergic reactions (e.g. anaphylaxis).

Research is also progressing in connection with the use of conjugates of transferrin and anticancer drugs as described in U.S. Pat. No. 5,108,987; 5,000,935; 4,895,714; and 4,886,780. The inventions described in these patents do not use antibodies. Instead, they use a protein found in normal human blood. This protein is transferrin, which delivers iron. Normal cells rarely require iron, but cancer cells require large amounts of iron to maintain their pathologically increased rates of metabolism Because cancer cells require more iron, they have transferrin receptors substantially permanently on their surfaces, whereas normal cells do not. These inventions exploits these receptors by administering anticancer drugs bonded to transferrin, which delivers the drugs substantially only to transferrin receptors on the surface of cancer cells.

Drug targeting spares normal cells, requires less drug, and significantly diminishes drug-toxicity. In contrast, when anticancer drugs are administered without being targeted, they kill normal cells as well as cancer cells. They are particularly toxic to cells of the immune system and to the system responsible for blood clotting. Thus, infections and bleeding are principal complications of chemotherapy in cancer patients. These complications require expensive services, hospitalizations, intensive care, and life-support systems, which are uncomfortable and expensive for the patient. These problems are largely preventable by using targeted delivery systems.

The problem of drug-toxicity consumes huge blocks of the time of doctors and nurses, and is responsible for much of the cost of cancer care. For example, it is commonly understood that about 70% of calls to oncologists relate to a problem of drug-toxicity. Today there is no satisfactory way to treat drug-toxicity, except to use less drug. Targeted delivery allows the use of less drug, because more of the administered drug is delivered specifically to cancer cells rather than being nonspecifically distributed around the body. In this sense, targeted delivery is like shooting with a rifle, while conventional delivery is like shooting with a shotgun. A solution to the problem of drug-toxicity will dramatically transform chemotherapy in cancer patients. It is a purpose of this invention to reduce such adverse effects of chemotherapy.

The problem of drug-resistance is equally as serious as the problem of drug-toxicity. This problem is typified by a patient diagnosed with cancer who is treated and responds with a symptomless remission that lasts many months, and who later sees the cancer returns in a form that no longer responds to any known drug. This scenario of drug-resistance is all too common. Yet today there is no satisfactory solution, except the use of larger amounts of more powerful drugs that in turn can cause serious drug-toxicity problems, often resulting in death. A solution to the problem of drug-resistance would significantly diminish the problem of drug-toxicity. Transferrin-targeted drug delivery can overcome the problem of drug-resistance. Thus, another purpose of the present invention is to resolve the issue of painful and expensive deaths from drug-resistant cancers.

The effectiveness of proteins conjugated with bio-affecting molecules has been demonstrated and is described in the US patents mentioned above. It has been determined; however, that the efficiency of such conjugates in treating stressed cells, such as cancer cells, is reduced by the presence of agglutinated conjugates or by the presence of conjugates of a bio-affecting molecule with protein fragments or with two or three protein molecules and is greatly enhanced when the protein to bio-affecting molecule ratio is closer to 1:1. Obtaining conjugates of higher efficiency has, in the past, been a slow, tedious and expensive process that requires separating a fraction of conjugate having the desired average ratio of bio-affecting molecule to protein from a larger sample comprising such molecules conjugated with protein fragments, with a plurality of proteins and proteins conjugated with a plurality of bio-affecting molecules. Using homogeneous protein-drug conjugates in which the protein component carries a predetermined number of bio-affecting molecules can more effectively kill both drug-resistant and drug-sensitive cancer cells. The past expense and inefficiency inherent in producing useful conjugates in a useful volume has been a problem for the commercialization of such conjugates and for their widespread use in medicine. There is a need for a substantially homogeneous drug-protein conjugate and for a method of making such a conjugate that is more efficient, more precise and less costly. It is one purpose of this invention to provide such a homogeneous conjugate made by a more efficient method.

### DESCRIPTION OF THE RELATED ART

The first report of transferrin receptors on human cancer cells was by Faulk and colleagues in 1980 (1). This was followed by many reports of transferrin receptors in different types of human cancers (2), as seen in the following Table.

| Tumor Studied | References |
|---|---|
| Breast | 1, 3 |
| Leukemia | 4, 5 |
| Lung | 6 |
| Brain | 7 |
| Liver | 8 |
| Bladder | 9 |
| Gastrointestinal | 10 |
| Ovary | 11 |
| Non-Hodgkin's lymphoma | 12 |
| Lymphoma/melanoma | 13, 14 |
| Nasopharyngeal | 15 |
| Cervix | 16 |

Transferrin Receptors on Normal and on Cancer Cells.

No single study has asked if all human cancers have up-regulated transferrin receptors, or if all normal cells have down regulated transferrin receptors, but data from many quarters suggest that the answer to both questions is yes. For example, immature erythrocytes (i.e., normoblasts and reticulocytes) have transferrin receptors on their surfaces, but mature erythrocytes do not (17). Circulating monocytes also do not have up-regulated transferrin receptors (18), and macrophages, including Kupffer cells, acquire most of their iron by a transferrin-independent method of erythrophagocytosis (19). In fact, in vivo studies indicate that virtually no iron enters the reticuloendothelial system from plasma transferrin (for review, see reference 20). Macrophage transferrin receptors are down regulated by cytokines such as gamma interferon (21), presumably as a mechanism of iron-restriction to kill intracellular parasites (22).

In resting lymphocytes, not only are transferrin receptors down regulated, but the gene for the transferrin receptor is not measurable (23). In contrast, stimulated lymphocytes up-regulate transferrin receptors in late G₁ (24). Receptor expression occurs subsequent to expression of the c-myc proto-oncogene and following up-regulation of IL-2 receptor (25), and is accompanied by a measurable increase in iron-regulatory protein binding activity (26), which stabilizes transferrin receptor mRNA (27). This is true for both T and B lymphocytes (28), and is an IL-2-dependent response (29).

Cell stimulation resulting in the up regulation of receptors for transferrin is known to result from stress experienced, for example, by cells invaded by a viral factor and by cancer cells.

Up-and-down regulation of transferrin receptors for normal and tumor cells has been shown by studies of antigen or lectin stimulation (ie., receptor up-regulation), and by studies of differentiation models (30-33) using retinoic acid (ie., receptor down-regulation). Base-line data from these experimental models suggest that these receptors are down regulated from the plasma membranes of most normal, adult, resting human cells (34). Exceptions are the circulatory barrier systems, which include the materno-fetal barrier with its transferrin receptor-rich syncytiotrophoblast (35); the blood-brain barrier with its transferrin receptor-rich capillary endothelial cells (36); and, the blood-testis barrier with its transferrin receptor-rich Sertoli cells (37).

Mechanism of Cell Killing by Transferrin-Drug Conjugates.

Transferrin-doxorubicin conjugates bind to plasma membranes by sequentially employing two reactions; initially the transferrin component is bound by transferrin receptors, after which the doxorubicin component is bound by the lipid bilayer, primarily by interacting with cardiolipin and charged phosphates (58). Thus, bound through protein and phospholipid receptors, the conjugates are positioned to activate signal transduction pathways by receptor dimerization, lateral mobility and cytoplasmic calcium mobilization (61).

One mechanism involved in the killing of tumor cells by transferrin-doxorubicin conjugates is the inhibition of plasma membrane redox enzymes, particularly the inhibition of NADH-oxidase (62). Inhibition of NADH-oxidase causes cell death (63), and doxorubicin is an efficient inhibitor of this enzyme (64,65). Transferrin-doxorubicin conjugates inhibit NADH-oxidase (66), as well as down-stream reactions initiated by NADH oxidation, such as loss of electrons and exchange of protons through the sodium-hydrogen antiport (67).

A second mechanism of cell killing by transferrin-doxorubicin conjugates involves the molecular control of transferrin receptors. For example, chelation of microenviromental iron initiates apoptosis in tumor cells but not in normal resting cells (68), and such chelation enhances significantly the cytotoxic effect of cytosine arabinoside (69). Drug-resistant cells are much more sensitive to iron restriction, due to their inability to stabilize transferrin receptor mRNA, and excess iron destabilizes transferrin receptor mRNA more effectively in drug-resistant than in drug-sensitive cells (70).

A third mechanism involves redox-active products of oxidative stress (71). For example, nitric oxide disassembles the iron-sulfur cluster, allowing iron-regulatory proteins to bind and protect iron-response elements (72). Hydrogen peroxide causes the same effect (i.e., up-regulation of transferrin receptors), but transferrin receptors are down regulated by the nitrosium ion, which causes nitrosylation of thiol groups within the iron-sulfur cluster (73). In summary, there are at least three mechanisms involved in the killing of cells by transferrin-doxorubicin conjugates.

Until now, the widespread treatment by such mechanisms of cells under stress and having up regulated transferrin receptors has effectively been blocked by the expense and time required to isolate a fraction of a protein-drug conjugate that contains a controlled ratio of protein to drug with substantially no dimers, polymers or fractions of protein from a reaction product that is likely to contain mostly such undesirable fractions. (As mentioned elsewhere, proteins such as transferrin that are agglutinated, fractionated, or the like will not interact correctly with transferrin receptors, if at all.) The inefficiency of the past process has made the treatment of cancer cells and of cells infected with a virus by these mechanisms economically unattractive.

Transferrin-Drug Conjugates in Laboratory Animals

The efficacy of transferrin-drug conjugates has been investigated in several animal models. For example, conjugates of transferrin with diphtheria toxin decrease xenografted gliomas in nude mice by 95% on day 14, and the gliomas did not recur by day 30 (74). Also, glutaraldehyde-prepared transferrin-doxorubicin conjugates have been found to rescue nude mice from death by human mesothelioma cells, significantly prolonging life compared to animals treated only with doxorubicin (75). In addition, transferrin has been coupled to herpes simplex thymidine kinase by using biotin-streptavidin technology, and these conjugates significantly prolonged life in nude mice inoculated with metastasizing K562 tumor cells (76). Finally, the maximum tolerated dose of human transferrin-doxorubicin conjugates in nude mice has been found to be 20 mg/kg (iv) for conjugates and only 8 mg/kg (iv) for free drug (41).

Transferrin-Drug Conjugates in Human Patients

There are two clinical reports of transferrin-drug conjugates. The first, published in 1990, was a preliminary study of seven acute leukemia patients treated intravenously with 1 mg/day of glutaraldehyde-prepared transferrin-doxorubicin conjugates for 5 days. With these low doses, there were no toxic effects and the number of leukemic cells in peripheral blood of the 7 patients decreased by 86% within 10-days following therapy (77). In addition, there was no extension of disease as assessed by examination of bone marrow biopsies before and after treatment.

The second, published by the NIH in 1997, involved 15 patients with recurrent brain cancers treated with thioether-bonded transferrin conjugates of a genetic mutant of diphtheria toxin (44). The conjugates were delivered by high-flow interstitial microinfusion, which has been shown to produce effective perfusion of radiolabeled transferrin in primate brains with minimal inflammatory responses (78). Magnetic resonance imaging revealed at least a 50% reduction in tumor volume in 9 of the 15 patients, including 2 cases of complete remission (44).

There is an unpublished clinical study of 23 patients with advanced ovarian cancer who were randomized into test (12 patients) and placebo (11 patients) groups. The test group received transferrin-doxorubicin conjugates equivalent to 1 mg doxorubicin per day on days 15 through 19 of monthly treatment cycles. A significant difference was revealed by Cox regression estimates of survival rates for patients treated with transferrin-doxorubicin conjugates when the time between diagnosis and randomization was 18 months.

Another unpublished study is a 22-year old male with metastatic disease from a sarcoma of his right atrium who was treated by conventional protocols without response. His lungs were filled with metastatic lesions when his physician father obtained an IND from the FDA for the use of transferrin-doxorubicin conjugates, and treatment was begun in August, 2000. By November, the lungs were substantially cleared of metastatic lesions, and by January there was no radiological evidence of tumor. He presently (August 2001) is active, receiving only transferrin-doxorubicin.

The targeted delivery of drugs has the advantage of increasing efficacy while using less drug, thereby decreasing toxicity and causing less damage to normal cells, all of which effectively decrease costs and increase the quality of patient care. Targeted delivery also avoids drug-resistance, which is activated by the non-specific entrance of drugs into cells (79). Because transferrin-drug conjugates enter cells specifically by employing a receptor-specific pathway (80,81), they are trafficked around drug-resistance mechanisms, such as efflux pumps in resistant cells.

It was reported in 1992 that transferrin-doxorubicin conjugates effectively kill multi-drug resistant cells (82). This finding was confirmed in 1993 (83), and was extended to several types of drug-resistant cells in 1994 (84), 1996 (85) and 2000 (86).

Preparation of Transferrin-Drug Conjugates.

A method for the preparation of transferrin-doxorubicin conjugates was published first in 1984 (38), following which there have been many reports of methods for the preparation of transferrin-drug conjugates, some of which are listed in the following Table.

| Transferrin Label | Method Used | References |
|---|---|---|
| Doxorubicin | Glutaraldehyde | 38, 39, 40 |
| Doxorubicin | Malcimide | 41 |
| Mitomycin C | Glutaryl Spacer | 42 |
| Neocarzinostatin | Succinimide | 43 |
| Diphtheria Toxin | Thioester | 44 |
| Chlorambucil | Maleimide | 45 |
| Paclitaxol | Glutaraldehyde | 46 |
| Daunorubicin | Glutaraldehyde | 47 |
| Titanium | Carbonate | 48 |
| Insulin | Disulfide | 49 |
| Gallium | Carbonate | 50 |
| Platinum | Methioninc | 51 |
| Saporin/ricin | Succinimide | 52 |
| Ruthenium | Bicarbonate | 53 |
| Growth Factor | Fusion Protein | 54 |
| HIV Protease | Recombinant | 55 |

Transferrin conjugates of doxorubicin can be prepared by using glutaraldehyde-mediated Schiff base formation (56,57), which forms an acid-resistant bond between epsilon-amino lysine groups of transferrin and the 3′amino position of doxorubicin. Such conjugates of doxorubicin can kill cancer cells through a plasma membrane-mediated mechanisms (for review, see reference 58). Although DNA intercalation is an established mechanism of cell death by doxorubicin, immobilized doxorubicin on carriers, such as dextran, activate plasma membrane-mediated mechanisms to kill cells (59,60). It thus appears that conjugates of doxorubicin with transferrin kill cells by activating plasma membrane-mediated mechanisms that involve both doxorubicin and transferrin receptors.

The ability of non-antibody proteins such as transferrin conjugated with anticancer drugs to target cancer cells and to kill drug-resistant cells efficiently has been found to depend on the molecular ratio of drug-to-transferrin. Excessive loading of a protein such as transferrin with bio-affecting molecules is believed to interfere with the protein's ability to dock with receptors. Under-loading of such a protein is believed to result in receptors being filled with proteins that do not carry drugs, a phenomenon known as blocking. Contemporary techniques for the preparation of transferrin-drug conjugates do not allow for the production of conjugates with predetermined ratios. Presently available procedures provide a heterogeneity of conjugates, including a large percentage that are either excessively loaded or that are under loaded. Isolation of a relatively small useful fraction from the presently available manufacturing procedures results in very low yields of clinically usable molecules and very high production costs. The expense involved causes the production and use of an otherwise effective cure for cancers and other conditions causing cells to undergo stress to be economically unattractive, thus denying the benefits of the material to most patients. There is a need for a high volume and lower cost method of making such conjugates.

### SUMMARY OF THE INVENTION

It is apparent that drugs combined with targeting agents have a generic possibility of changing how drugs are delivered, as well as a specific possibility of changing how drugs are delivered to cancer patients. However, it is a problem that it hitherto has not been possible to synthesize large amounts of homogeneous conjugates with predetermined and consistent numbers of drug molecules per molecule of protein. This and other problems with known methods for making and using conjugates of proteins, such as transferrin, ceruloplasmin, vitamins, vitamin binding proteins, hormones, cytokines, low density lipoproteins, and growth factors, with anticancer drugs (e.g. cytostatic or cytotoxic agents, photosensitizers, heat sensitizers, and apoptosis inducing compounds) are solved by the present invention.

In one aspect the present invention comprises a material for treating diseased cells wherein the material includes a substantially homogeneous and predetermined ratio of a protein capable of binding with receptors up regulated by cells in response to the disease conjugated with a bio-affecting molecule.

In yet another aspect, the invention comprises forming a bio-affecting molecule-linker moiety wherein the linker is capable of further reacting with a protein and linking the moiety to a protein wherein the protein can bind with cells stressed by disease and wherein the bio-affecting molecule treats the cell or makes the cell visible to imaging techniques. In still another aspect, the invention comprises a method for treating cells having a relatively high attraction for the protein by contacting such cells with the material

### DETAILED DESCRIPTION OF THE INVENTION

The presently preferred method comprises adding a bio-affecting material, such as an anticancer drug, to a linker such that there is a controlled ratio of bio-affecting material connected to each linker molecule. The drug-linker material is added to a protein such as transferrin, vitamins, vitamin binding proteins, hormones, cytokines, low-density lipoproteins, and growth factors in amounts to achieve a desired molar ratio. In the presently preferred process the linker is glutaraldehyde. Glutaraldehyde was selected as a linker because its presents only two reaction sites and because its reaction kinetics favor the attachment of only one bio-affecting molecule to each linker molecule. Excess glutaraldehyde may be scavenged with, for example, ethanolamine following formation of the conjugate.

The present invention relates to homogeneous conjugates with a predetermined and consistent number of antitumor agent or other bio-affecting molecule per molecule of protein targeting agent. The targeting agents according to the present invention include but are not limited to transferrin, ceruloplasmin, vitamins, vitamin binding proteins, hormones, cytokines, low density lipoproteins, and growth factors. The anti-tumor agent or other bioaffecting molecule includes but is not limited to cytotoxic agents such as doxorubicin, methotrexate, vincristin, daunomycin, 6-mercaptopurine, cytosine arabinoside, and cyclo phosphamide, heat sensitizers such as hematophorphyrine and low-dose verapamil, apoptosis inducing compounds such as deferoxamine, photosensitizers such as porfimer sodium, metatetrahydroxyphenylchlorin, and hematophorphyrin derivatives, and imaging materials such as isotopes, fluorescent molecules and radio opaqing materials Preferably the targeting agent is transferrin and the anti-tumor agent is doxorubicin.

Conjugates which include imaging materials are described in U.S. Pat. No. 4,895,714, issued on Jan. 23, 1990, and 5,000,935, issued in Mar. 19, 2001, which are hereby incorporated by reference. Suitable isotopes include but are not limited to iodine, gallium, indium, and yttrium, preferably ¹²⁵I., ¹³¹I, ¹¹¹In, ⁹⁰Y, and ⁶⁷Ga.

The invention also relates to efficient and economical methods for preparing substantially homogeneous conjugates having a predetermined and consistent number of antitumor agents or other bio-affecting molecules per molecule of protein targeting agent. This process substantially reduces, and in most cases virtually eliminates the production of polymers and dimers of transferrin or aggregates of transferrin drug conjugates, thus yielding a narrow range of drug-protein ratios. The present invention substantially decreases production costs and increases efficiency while increasing the effectiveness of the conjugate in medical applications. The substantially homogeneous conjugates according to the present invention result from a process beginning with the formation of reactive drug-linker complexes. In this illustration of the process the bio-affecting material is doxorubicin and the linker is glutaraldehyde. It will be understood that other bio-affecting materials and linkers will also be useful.

The synthesis of large amounts of homogeneous transferrin-doxorubicin conjugates with predetermined molecular ratios was done stoichiometrically by employing the only amino group of doxorubicin (DOX), which is at the 3′ amino position, to react with one of the two reactive groups on glutaraldehyde (GLU). Thus, the first step was drop-wise addition of a saline solution of DOX into a saline solution of GLU containing a solvent such as DMSO or another suitable cryopreservative, to a final concentration of a 1:1 molar ratio of DOX-to-GLU. The resulting solution of DOX-GLU was stirred three hours at room temperature in the dark.

The molarities of DOX and GLU were the same in the above reaction in order to produce a final solution of DOX-GLU that contains neither free DOX nor free GLU. However, there is the possibility of free GLU in solution if one GLU reacts with two DOX to produce DOX-GLU-DOX but this possibility is minimized by the mass action kinetics generated by drop-wise addition of monovalent DOX into the solution of bivalent GLU. The volumes of these reactants are not restricted, so large amounts of homogeneous DOX-GLU can be prepared.

The second step in the conjugation reaction was drop-wise addition of DOX-GLU into a saline solution of transferrin (TRF). The TRF can be either iron-free (apo-transferrin) or iron-saturated (holo-transferrin). The desired molar ratio of DOX to TRF was obtained by appropriately adjusting the volume of TRF. The resulting solution of TRF-GLU-DOX was stirred for 20 hours at room temperature in the dark. Unlike the reaction of DOX with GLU, the reaction of DOX-GLU with TRF is not restricted to one binding site, for the GLU component of DOX-GLU can react with any one of several epsilon-amino lysine groups in the TRF molecule.

The number of DOX molecules bound to TRF was determined in the second step. For example, if the starting ratio of DOX-GLU to TRF was 7.2:1.0, the final solution of TRF-GLU-DOX would have contained 2.5 molecules of DOX per molecule of TRF. However, if the starting ratio of DOX-GLU to TRF was 4.0:1.0, the final solution of TRF-GLU-DOX would have contained 1.4 molecules of DOX per molecule of TRF. Similarly, if the starting ratio of DOX-GLU to TRF was 2.5:1.0, the final solution of TRF-GLU-DOX would have contained 0.9 molecules of DOX per molecule of TRF. In this way, large amounts of TRF-GLU-DOX with predetermined ratios of DOX-to-TRF can be provided according to the need.

Further steps in the conjugation reaction were the addition of ethanolamine or another substance suitable for scavenging any excess linker, followed by centrifugation and dialysis. Although reactions with DOX and TRF theoretically consume all of the GLU, ethanolamine was added to the final reaction mixture to bind any available GLU. This reaction was allowed to continue for 30 minutes in the dark. The final solution was centrifuged at 2000 rpm for 10 minutes, dialyzed twice for 6 hours in a 100-fold excess of saline and three times in the same excess of Hepes buffered saline, and the resulting TRF-GLU-DOX conjugates were ready for use.

Biochemical Characterization of the Conjugates:

By using HPLC and polyacrylamide gel electrophoresis as described in (39), the homogeneity of TRF-GLU-DOX conjugates can be determined. Also, by using spectrophotometry as described in (89), the molecular ratio of DOX-to-TRF can be determined. These techniques repeatedly have revealed a consistent homogeneity of the TRF-GLU-DOX conjugates. In addition, chromatography is not required in the preparation of these conjugates, because there are no aggregates or fragments. This allows for the preparation of large volumes of homogeneous transferrin-drug conjugates, which increases yields and decreases costs.

The expenses caused by losses of TRF and DOX in other types of transferrin-drug conjugates have been an impediment to their use. For example, yields of DOX and TRF are decreased by using procedures such as thiolation (44) that alter the drug and/or protein. Yields also are decreased by using solvent systems (86) and by chromatography used to prepare acid-stable and acid-labile linkages (41). The GLU bond between DOX and TRF is acid-stable (89), and yields of DOX and TRF in TRF-DOX conjugates prepared according to this invention are high. Indeed, compared to other procedures (38, 39, 40), the yield for TRF is nearly doubled (90% vs 50%), and the yield for DOX is increased 5-fold.

None of the previously known approaches to the preparation of transferrin-doxorubicin conjugates are capable of producing large amounts of homogeneous conjugates with predetermined ratios of the number of drug molecules per molecule of transferrin. In addition, the other approaches employ chromotography to eliminate aggregates and to harvest fractions that are enriched in homogeneous conjugates. These procedures decrease yields, increase costs, and lack the ability to predetermine molecular ratios.

Another procedure would be to mix one milliliter of transferrin (0.5 mM) with one milliliter of deferoxamine (8.5 mM) in 150 mM sodium chloride for 4 minutes, and then add one milliliter of 21.5 mM glutaraldehyde in 150 mM sodium chloride and mix 4 minutes. The preceding reaction is a coupling procedure, which is stopped by the addition of 0.8 milliliters of 37.2 mM ethanolamine in 150 mM sodium chloride and 10 mM Hepes buffer (pH8) and vortexed for 4 minutes. The mixture (3.8 milliliters) then is transferred to dialysis tubing (molecular weight cutoff of 12,000-14,000), and dialyzed against 0.5 liters of Hepes-buffered saline in the dark at 5° C. for 3 hours. The dialysis should be repeated at least once with fresh Hepes-buffered saline. The mixture then is centrifuged at 1600 g for 10 minutes at 4° C. and the supernatant is chromatographed at a flow rate of 22 milliliters per hour on a 2.6×34 cm column of Sepharose CL-4B, previously equilibrated in Hepes-buffered saline and calibrated at 5° C. with blue dextran, transferrin and cytochrome C. Elution from the column is monitored at 280 nm, and 3.8 milliliter fractions are collected. The concentration of transferrin and deferoxamine in each fraction is calculated by successive approximation from standard curves from transferrin and deferoxamine, determined by using 280 nm for transferrin and 356 nm for deferoxamine. With minor modifications, this coupling procedure can be used to prepare targeting protein conjugates of other iron chelating drugs, such as protein conjugates of hydrophobic reversed siderophores.

Characterizing the Conjugates

After the pure drug-protein conjugates are isolated, they are characterized by polyacrylamide gel electrophoresis to determine their molecular weight, and the number of drug molecules per protein molecule is determined. The exact number of drug molecules per transferrin molecule can be determined, using any suitable technique including but not limited to spectrophotometric techniques. A functional drug: protein ratio is between about 0.1:1.0 to 3.0:1.0 (Berczi et al., Arch Biochem Biophys 1993; 300:356). The conjugates are checked to determine if they bind to receptors on the surface of tumor cells, and to determine if the conjugates kill cancer cells but not normal cells. Only conjugates that bind to cancer cells and not to normal cells are selected for toxicity tests using drug-sensitive and drug-resistant cancer cells. The binding studies can be done by using flow cytometry or any other suitable method, and the killing studies can be done by using microculture techniques to determine the concentration of free drug required to kill 50% of a culture of cancer cells compared to the concentration of drug in the drug-protein conjugates required to kill the same number of cancer cells. When testing the heat sensitizer conjugates, the toxicity test is done by using the MIT tetrazolium colorimetric assay (Visitica et al., Cancer Res 1994; 51: 2515). These toxicity tests determine the most potent transferrin sensitizer ratio and the optimum concentration of conjugate for maximum heat sensitization of drug sensitive and drug resistant cells. Approximately 10-fold more free drug compared to drug in the drug-protein conjugate is required to kill the same number of cells.

While the above description refers to transferrin as being the delivery protein, it is known that other proteins exist in the body which are capable of binding to receptor sites on cells. If such a receptor site is activated in cancer cells and is inactive in normal cells, then any protein or other molecule (i.e., ligand) that binds to such a receptor site can be used to deliver the drugs used in the present invention. An example of such a binding protein is transcobalamin, which delivers vitamin B12 to transcobalamin receptors on cells, including cancer cells (Seetheram, Ann Rev Nutr 1999; 19:173). Low density lipoprotein is another ligand that has been conjugated to the photosensitizer chlorin and targeted to low density lipoprotein receptors on retinoblastoma cells (Schmidt-Erfurth et al., Brit J Surg 1997; 75:54).

After the drug-protein conjugate has been prepared, purified, characterized and validated for cellular binding and killing properties, and, when the binding and killing experiments show that the conjugate binds to and kills cancer but not normal cells, the conjugate is then aliquoted and sterilized. The sterilization process can be done by any suitable method including but not limited to exposure to irradiation, such as by using a cesium irradiator, or by using Millipore filtration techniques.

According to a further aspect of the present invention, there is provided a reagent kit for the treatment of tumors, comprising iron-bearing transferrin and a homogeneous conjugate with a predetermined and consistent ratio of antitumor agent molecules per molecule of transferrin. The patient's normal cells which have transferrin receptors may be protected against the effects of the conjugate by saturating these receptors with the iron-bearing transferrin before administration of the homogeneous conjugate.

The present invention also provides a process for determining the susceptibility of tumor cells to anti-tumor agents, comprising administering separately to portions of said tumor cells homogeneous conjugates of transferrin with a number of different anti-tumor agents. A reagent kit comprising a number of such different conjugates may be provided for this purpose. Because the homogeneous conjugates of the present invention are taken up extremely rapidly by tumor cells, cells may be tested against a range of homogeneous conjugates of a targeting protein with different anti-tumor agents. Such a process increases the efficiency of any subsequent chemotherapy and enables it to be started quickly after isolation of the tumor cells.

As used in the present document, the term “substantially homogeneous conjugates” means that the conjugates can be used without further purification to remove protein diners, polymers or aggregates. In other words, little or no protein dimers, polymers or aggregates are present.

The substantially homogeneous conjugates according to the present invention are administered to an animal in an effective amount. In treating cancer, an effective amount includes an amount effective to: reduce the size of a tumor; slow the growth of a tumor; prevent or inhibit metastases; or increase the life expectancy of the affected animal. The present invention provides for a method of treating a variety of cancers including but not limited to leukemia, breast cancer, ovarian cancer, pancreatic cancer, lung cancer, bladder cancer, gastrointestinal cancer, nasopharyngeal cancer, cervical cancer, myeloma, lymphoma/melanoma, glioma, or astrocytoma. The dosage for the homogeneous conjugates can be determined taking into account the age, weight and condition of the patient and the pharmacokinetics of the anti-tumor agent. The amount of the homogeneous conjugate required for effective treatment will be less than the amount required using the anti-tumor agent alone. For example, the dosage of a conjugate of transferrin-doxorubicin is expected to be between 0.5-50 mg per 28 day period for a 150 pound (68 kg) person. The dosage can be divided and administered as smaller doses at varying intervals during the 28 day period.

The pharmaceutical compositions of the invention can be administered by a number of routes, including but not limited to orally, topically, rectally, ocularly, vaginally, by the pulmonary route, for instance, by use of an aerosol, or parenterally, including but not limited to intramuscularly, subcutaneously, intraperitoneally, intra-arterially or intravenously. The compositions can be administered alone, or can be combined with a pharmaceutically-acceptable carrier or excipient according to standard pharmaceutical practice. For the oral mode of administration, the compositions can be used in the form of tablets, capsules, lozenges, troches, powders, syrups, elixirs, aqueous solutions and suspensions, and the like. For parenteral administration, sterile solutions of the homogeneous conjugate are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. For pulmonary administration, diluents and/or carriers will be selected to be appropriate to allow the formation of an aerosol. It is preferred that the conjugate of the present invention be administered parenterally, i.e. intravenously or intraperitoneally, by infusion or injection.

Preferred embodiments of the present invention are described below. It will be apparent to those of ordinary skill in the art after reading the following description that modifications and variations are possible, all of which are intended to fall within the scope of the claims.

### EXAMPLE 1

Preparation of a Homogeneous Transferrin-Doxorubicin Conjugate

The synthesis of large amounts of homogeneous transferrin-doxorubicin conjugates with predetermined molecular ratios was done stoichiometrically by employing the only amino group of doxorubicin (DOX), which is at the 3′ amino position, to react with one of the two reactive groups on glutaraldehyde (GLU). Thus, the first step was drop-wise addition of a saline solution of DOX into a saline solution of GLU containing a solvent such as DMSO to a final concentration of a 1:1 molar ratio of DOX-to-GLU. The resulting solution of DOX-GLU was stirred three hours at room temperature in the dark.

The molarities of DOX and GLU were the same in the above reaction in order to produce a final solution of DOX-GLU that contains neither free DOX nor free GLU. However, there is the possibility of free GLU in solution if one GLU reacts with two DOX to produce DOX-GLU-DOX, but this possibility is minimized by the mass action kinetics generated by drop-wise addition of monovalent DOX into the solution of bivalent GLU. The volumes of these reactants are not restricted, so large amounts of homogeneous DOX-GLU can be prepared.

The second step in the conjugation reaction was drop-wise addition of DOX-GLU into a saline solution of transferrin (TRF). The TRF can be either iron-free (apo-transferrin) or iron-saturated (holo-transferrin). The desired molar ratio of DOX to TRF was obtained by appropriately adjusting the volume of TRF. The resulting solution of TRF-GLU-DOX was stirred for 20 hours at room temperature in the dark Unlike the reaction of DOX with GLU, the reaction of DOX-GLU with TRF is not restricted to one binding site, for the GLU component of DOX-GLU can react with any one of several epsilon-amino lysine groups in the TRF molecule.

The number of DOX molecules bound to TRF was determined in the second step. For example, if the starting ratio of DOX-GLU to TRF was 7.2:1.0, the final solution of TRF-GLU-DOX would have contained 2.5 molecules of DOX per molecule of TRF. However, if the starting ratio of DOX-GLU to TRF was 4.0:1.0, the final solution of TRF-GLU-DOX would have contained 1.4 molecules of DOX per molecule of TRF. Similarly, if the starting ratio of DOX-GLU to TRF was 2.5:1.0, the final solution of TRF-GLU-DOX would have contained 0.9 molecules of DOX per molecule of TRF. In this way, large amounts of TRF-GLU-DOX with predetermined ratios of DOX-to-TRF can be provided according to the need.

Further steps in the conjugation reaction were the addition of ethanolamine, followed by centrifugation and dialysis. Although reactions with DOX and TRF theoretically consume all of the GLU, ethanolamine was added to the final reaction mixture to bind any available GLU. This reaction was allowed to continue for 30 minutes in the dark. The final solution was centrifuged at 2000 rpm for 10 minutes, dialyzed twice for 6 hours in a 100-fold excess of saline and three times in the same excess of Hepes buffered saline, and the resulting TRF-GLU-DOX conjugates were ready for use. Biochemical Characterization of the Conjugates:

By using HPLC and polyacrylamide gel electrophoresis as described in (39), the homogeneity of TRF-GLU-DOX conjugates can be determined. Also, by using spectrophotometry as described in (89), the molecular ratio of DOX-to-TRF can be determined. These techniques repeatedly have revealed a consistent homogeneity of the TRF-GLU-DOX conjugates. In addition, chromatography is not required in the preparation of these conjugates, because there are no aggregates or fragments. This allows for the preparation of large volumes of homogeneous transferrin-drug conjugates, which increases yields and decreases costs.

The expenses caused by losses of TRF and DOX in other types of transferrin-drug conjugates have been an impediment to their use. For example, yields of DOX and TRF are decreased by using procedures such as thiolation (44) that alter the drug and/or protein. Yields also are decreased by using solvent systems (86) and by chromatography used to prepare acid-stable and acid-labile linkages (41). The GLU bond between DOX and TRF is acid-stable (89), and yields of DOX and TRF in TRF-DOX conjugates prepared according to this invention are high. Indeed, compared to other procedures (38, 39, 40), the yield for TRF is nearly doubled (90% vs 50%), and the yield for DOX is increased 5-fold.

None of the previously known approaches to the preparation of transferrin-doxorubicin conjugates are capable of producing large amounts of substantially homogeneous conjugates with predetermined ratios of the number of drug molecules per molecule of transferrin In addition, the other approaches employ chromatography to eliminate aggregates and to harvest fractions that are enriched in homogeneous conjugates. These procedures decrease yields, increase costs, and lack the ability to predetermine molecular ratios.

In Vitro Characterization of the Conjugates:

Conjugates of TRF-GLU-DOX prepared according to this invention have the ability to bind and kill cancer cells but not normal cells. By using flow cytometry as described in (39), these conjugates have been shown to bind cultured human cancer cells and not normal peripheral blood lymphocytes. Also, by using cell culture techniques as described in (39), the TRF-DOX conjugates have been shown to kill cultured human cancer cells but not normal cells. These procedures also serve as quality controls for the homogenous TRF-GLU-DOX conjugates described herein.

The TRF-GLU-DOX conjugates described in this patent also have the ability to kill drug-resistant cancer cells. Earlier experimental data indicated that other conjugates of TRF with anticancer drugs can kill multi-drug resistant human cancer cells by binding transferrin receptors (82), and that such resistant cells have been shown to have more transferrin receptors on their surfaces than do drug-sensitive cells (90). Though presently unpublished and unreported, the TRF-GLU-DOX conjugates described herein have been found to uniformly bind and kill drug-resistant cells. Thus, homogeneous TRF-GLU-DOX conjugates with predetermined molecular ratios, such as those described herein, provide clinically useful molecules for killing both drug-resistant and drug-sensitive cancer cells by uniformly and consistently binding transferrin receptors.

In Vivo Characterization of the Conjugates:

Nude mice xenografted with human drug-resistant human mesothelioma cancer cells survived significantly longer when they were treated with TRF-DOX conjugates than when they were treated with free DOX (75), providing proof-of-principle that transferrin-drug conjugates kill drug-resistant human cancer cells in a mouse mode. However, these results are dependent on the ability to produce large amounts of homogeneous TRF-DOX conjugates containing a predetermined number of DOX molecules per molecule of TRF.

In unpublished experiments using TRF-DOX conjugates prepared as described herein, nude mice xenografted with lethal doses of drug-sensitive and drug-resistant human cancer cells survived significantly longer when treated with TRF-DOX conjugates than when treated with placebo (i.e., albumin), unconjugated TRF or free DOX. In these experiments, mice with drug-resistant tumors received the same dose of TRF-DOX as mice with drug-sensitive tumors.

It will be apparent to one of ordinary skill that conjugates can be made with various linkers and ratios of linker to bio-affecting molecule, all of which are intended to be within the scope of the appended claims. It will also be apparent that the method of making such conjugates will also apply when the conjugates include radioisotopes for imaging or radio-opaqing materials either instead of or in addition to bio-affecting molecules. The use of the homogeneous conjugates of the present invention in imaging tumors and in treating tumors with radioisotopes is intended to be within the scope of the appended claims.

### REFERENCES

(References in bold type are from Dr. Faulk's laboratory)

1. Faulk W P, Hsi B L and Stevens P J. Transferrin and transferrin receptors in carcinoma of the breast. Lancet 1980; ii: 390-392

2. Faulk W P, Harats H and Berczi A. Transferrin receptor growth control in normal and abnormal cells. In: Oxidoreduction at the Plasma Membrane. Vol 1. (eds., F L Crane, J D Morre and H Low) CRC Press, Boca Raton, Fla., 1990; pp. 205-224.

3. Yang D C, Wang F, Elliott R L and Head J F. Expression of transferrin receptor and ferritin H-chain mRNA are associated with clinical and histopathological prognostic indicators in breast cancer. Anticancer Res 2001; 21: 541-549.

4. Yeh C J G, Taylor C G and Faulk W P. Targeting of cytotoxic drugs by transferrin receptors: Selective killing of acute myelogenous leukemia cells. Protides Biol Fluids 1984; 32: 441-444.

5. Barnett D, Wilson G A, Lawrence A C and Buckley G A Transferrin receptor expression in the leukaemias and lymphoproliferative disorders. Clin Lab Haematol 1987; 9: 361-70.

6. Whitney J F, Clark J M, Griffin T W, Gautam S and Leslie K O. Transferrin receptor expression in nonsmall cell lung cancer. Histopathologic and clinical correlates. Cancer 1995; 76: 20-25.

7. Recht L, Torres C O, Smith T W, Raso V and Griffin T W. Transferrin receptor in normal and neoplastic brain tissue: implications for brain-tumor immunotherapy. J Neurosurg 1990; 72: 941-945.

8. Sciot R, Paterson A C, van Eyken P, Callea F, Kew M C and Desmet V J. Transferrin receptor expression in human hepatocellular carcinoma: an immunohistochemical study of 34 cases. Histopathol 1988; 12: 53-63

9. Seymour G J, Walsh M D, Lavin M F, Strutton G and Gardiner R A Transferrin receptor expression by human bladder transitional cell carcinomas. Urol Res 1987; 15: 341-344.

10. Lindholm M L, Lindberg L A, Vilja P, Puolakka V M, Nordling S, Schroder T and Schroder J. Expression of the human transferrin receptor in subrenal capsule assay in the mouse. J Surg Oncol 1988; 38: 57-62.

11. Hereiz H A and Bayoumi F A Evaluation of diagnosis of ovarian malignancy using immunohistochemical technique. J Egyptian Public Hlth Assoc 1992; 67: 697-707.

12. Medeiros L J, Picker L J, Horning S J and Warnke R A Transferrin receptor expression by non-Hodgkin's lymphomas. Correlation with morphologic grade and survival Cancer 1988; 61: 1844-185.

13. Yeh C J G, Taylor C and Faulk W P. Transferrin binding by peripheral blood mononuclear cells in human lymphomas, myelomas and leukemias. Vox Sanguinis 1984; 46: 217-223.

14. Soyer H P, Smolle J, Tome R and Kerl H. Transferrin receptor expression in normal skin and in various cutaneous tumors. J Cutaneous Pathol 1987; 14: 1-5.

15. Pannccio M, Zalcberg J R, Thompson C H, Leyden J M, Sullivan J R, Lichtenstein M and McKenzie I F. Heterogeneity of the human transferrin receptor and use of anti-transferrin receptor antibodies to detect tumors in vivo. Immunol & Cell Biol 1987; 65: 461-472.

16. Farley J, Loup D, Nelson M, Miller M J, Taylor R and Gray K Transferrin in normal and neoplastic endocervical tissues: distribution and receptor expression. Analyst & Quant Cytol & Histol 1998; 20: 238-249.

17. Lesley J, Hyman R, Schulte R and Trotter J. Expression of transferrin receptor on murine hematopoietic progenitors. Cell Immunol 1984; 83: 14-25.

18. Testa U, Pelosi E and Peschle C. The transferrin receptor. Crit Rev Oncogen 1993; 4: 241-276.

19. Bothwell T A, Charlton R W, Cook J D and Finch C A. Iron Metabolism in Man, Blackwell Scientific, Oxford, 1979.

20. Ponka P and Lok C N. The transferrin receptor: role in health and disease. Int J Biochem Cell Biol 1999; 31: 1111-1137.

21. Hamilton T A, Gray P W and Adams D O. Expression of the transferrin receptor on murine peritoneal macrophages is modulated by in vitro treatment with interferon gamma Cell Immunol 1984; 89: 478-488.

22. Byrd T F and Horowitz M A. Interferon gamma-activated human monocytes downregulate transferrin receptors and inhibits the intracellular multiplication of Legionella pneumophila by limiting the availability of iron. J Clin Invest 1989; 83: 1457-1465.

23. Kronke M, Leonard W, Depper J M and Greene W C. Sequential expression of genes involved in human T lymphocyte growth and differentiation J Exp Med 1985; 161: 1593-1598.

24. Galbraith R M and Galbraith G M. Expression of transferrin receptors on mitogen-stimulated human peripheral blood lymphocytes: relation to cellular activation and related metabolic events. Immunology 1983; 133: 703-710.

25. Neckers L M and Cossman J. Transferrin receptor induction in mitogen-stimulated human T lymphocytes is required for DNA synthesis and cell division and is regulated by interleukin 2. Proc Nat Acad Sci USA 1983; 80: 3494-3498.

26. Testa U, Kuhn L, Petrini M, Quaranta M T, Pelosi E and Peschle C. Differential regulation of iron regulatory element-binding protein(s) in cell extracts of activated lymphocytes versus monocytes-macrophages. J Biol Chem 1991; 266: 3925-3930.

27. Seiser C, Texieira S and Kuhn L C. Interleukin-2-dependent transcriptional and post-transcriptional regulation of transferrin receptor mRNA J Biol Chem 1993; 268: 13,074-13,080.

28. Neckers L M, Yenokida G and James S P. The role of the transferrin receptor in human B lymphocyte activation. J Immunol 1984; 133: 2437-2441.

29. Neckers L M and Trepel J B. Transferrin receptor expression and the control of cell growth. Cancer Invest 1986; 4: 461-470.

30. Yeh C J G, Papamichail M and Faulk W P. Loss of transferrin receptors following induced differentiation of HL-60 promyelocytic leukemia cells. Exper Cell Res 1982; 138: 429-431.

31. Barker K A and Newburger P E. Relationships between the cell cycle and the expression c-myc and transferrin receptor genes during induced myeloid differentiation. Exper Cell Res 1990; 186: 1-5.

32. Klausner R D, Rouault T A and Harford J B. Regulating the fate of mRNA: the control of cellular iron metabolism Cell 1993; 72: 19-28.

33. Haile D J. Regulation of genes of iron metabolism by the iron-response proteins. Am J Med Sciences 1999; 318: 230-240.

34. Gatter K C, Brown G, Trowbridge I S, Woolston R E and Mason D Y. Transferrin receptors in human tissues: their distribution and possible clinical relevance. J Clin Pathol 1983; 36: 539-545.

35. Faulk W P and Hunt J S. Human placentae: view from an immunological bias. Am J Reprod Immunol 1990; 21: 108-113.

36. Broadwell R D, Baker-Caims B J, Friden P M, Oliver C and Villegas J C. Transcytosis of protein through the mammalian cerebral epithelium and endothelium III. Receptor mediated transcytosis through the blood-brain barrier of blood-borne transferrin and antibody against transferrin receptor. Exp Neurol 1996; 142: 47-65.

37. Sylvester S R and Griswold M D. The testicular iron shuttle: A “nurse” function of the Sartoli cells. J Androl 1994; 15: 381-385.

38. Yeh C J G and Faulk W P. Killing of human tumor cells in culture with doxorubicin conjugates of human transferrin Clin Immunol Immunopath 1984; 32:1-11.

39. Berczi A, Barabas K, Sizensky J A and Faulk W P. Doxorubicin conjugates of human transferrin bind transferrin receptors and kill K562 and HL60 cells. Arch Biochem Biophys 1993; 300: 356-363.

40. Lai B T, Gao J P and Lanka K W. Mechanism of action and spectrum of cell lines sensitive to a doxorubicin-transferrin conjugate. Cancer Chemother & Pharmacol 1998; 41: 155-160.

41. Kratz F, Beyer U, Roth T, Tarasova N, Collery P, Lechenault F, Cazabat A, Schumacher P, Unger C and Falken U. Transferrin conjugates of doxorubicin synthesis, characterization, cellular uptake, and in vitro efficacy. J Pharm Sciences 1998; 87: 338-346.

42. Tanaka T, Kaneo Y and Miyashita M. Synthesis of transferrin-mitomycin C conjugate as a receptor-mediated drug targeting system. Biol Pharm Bull 1996; 19: 774-777.

43. Sasaki K, Kongo Y, Kato J, Kondo H and Niitsu Y. Intracellular metabolism and cytotoxicity of transferrin-neocarzinostatin conjugates of differing molar ratios. Jpn J Cancer Res 1993; 84: 191-196.

44. Laske D W, Youle R J and Oldfield E H. Tumor regression with regional distribution of the targeted toxin TF-CRM107 in patients with malignant brain tumors. Nature Med 1997; 3: 1362-1368.

45. Beyer U, Roth T, Schumacher P, Maier G, Unold A, Frahm A W, Fiebig H H, Unger C and Kratz F. Synthesis and in vitro efficacy of transferrin conjugates of the anticancer drug chlorambucil. J Med Chem 1998; 41: 2701-2708.

46. Bicamumpaka E and Page M. In vitro cytotoxicity of paclitaxel-transferrin conjugate on H69 cells. Oncol Reports 1998; 5: 1381-1383.

47. Lemieux P, Page M and Noel C. In vivo cytotoxicity and antineoplastic activity of a transferrin-daunorubicin conjugate. In Vivo 1992; 6: 621-627.

48. Guo M, Sun H McArdle H J, Gambling L and. Sadler P J. Ti(IV) uptake and release by human serum transferrin and recognition of Ti(IV)-transferrin by cancer cells: understanding the mechanism of action of the anticancer drug titanocene dichloride. Biochem 2000; 39: 10023-10033.

49. Shah D and Shen W C. Transcellular delivery of an insulin-transferrin conjugate in enterocyte-like Caco-2 cells. J Pharm Sciences 1996; 85: 1306-1311.

50. Drobyski W R, Ul-Haq R, Majewski D and Chitambar C R. Modulation of in vitro and in vivo T-cell responses by transferrin-gallium and gallium nitrate. Blood 1996; 88: 3056-3064.

51. Hoshino T, Misaki M, Yamamoto M, Shimizu H, Ogawa Y and Toguchi H. In vitro cytotoxicities and in vivo distribution of transferrin-platinum(II) complex J Pharm Sciences 1995; 84: 216-221.

52. Ippoliti R, Ginobbi P, Lendaro E, D'Agostino I, Ombres D, Benedetti P A, Brunori M and Citro G. The effect of monensin and chloroquine on the endocytosis and toxicity of chioneric toxins. Cell Mol Life Sci 1998; 54: 866-875.

53. Kratz F, Hartmann F, Keppler B and Messor L. The binding properties of two antitumor ruthenium(III) complexes to apotransferrin. J Biol Chem 1994; 269: 2581-2588.

54. Park E, Starzyk R M, McGrath J P, Lee T, George J, Schutz A J, Lynch P and Putney S D. Production and characterization of fusion proteins containing transferrin and nerve growth factor. J Drug Targeting 1998; 6: 53-64.

55. Ali S A, Joao H C, Hammerschmid F, Eder J and Steinkasserer A Transferrin Trojan Horses as a rational approach for biological delivery of therapeutic peptide domains. J Biol Chem 1999; 274: 24066-24073.

56. Peters K and Richards F M. Chemical cross-linking: reagents and problems in studies of membrane structure. Annu Rev Biochem 1977; 46: 523-551.

57. Rhodes J. Evidence for an intercellular covalent reaction essential in antigen-specific T cell activation. J Immunol 1989; 143: 1482-1489.

58. Tritton T R. Cell surface actions of doxorubicin. Pharmacol & Therapeutics 1991: 49: 293-309.

59. Maestre N, Tritton T R, Laurent G and Jaffrezou J P. Cell surface-directed interaction of anthracyclines leads to cytotoxicity and nuclear factor kappaB activation but not apoptosis signaling. Cancer Res 2001; 61: 2558-2561.

60. Fong W F, Lam W, Yang M and Wong J T-F. Partial synergism between dextran-conjugated doxorubicin and cancer drugs on the killing of multidrug resistant KB-VI cells. Anticancer Res 1996; 16: 3773-3778.

61. Sainte-Marie J, Lafont V, Pecheur E I, Favero J, Philippot J R and Bienvenue A. Transferrin receptor functions as a signal-transduction molecule for its own recycling via increases in the internal Ca++ concentration. Euro J Biochem 1997; 250: 689-697.

62. Crane F L, Low H, Sun I L, Morre D J and Faulk W P. Interaction between oxidoreductase, transferrin receptor and channels in the plasma membrane. In: Growth Factors from Genes to Clinical Applications (eds, V R Sara, K Hall and H Low) Raven Press, New York, 1990; pp. 228-239.

63. Sun I L, Navas P, Crane F L, Morre D J and Low H. Diferric transferrin reductase in the plasma membrane is inhibited by doxorubicin. Biochem Int 1987; 14: 119-127.

64. Faulk W P, Harats H, McIntyre J A, Berczi A, Sun I L and Crane F L. Recent advances in cancer research: Drug targeting without the use of monoclonal antibodies. Am J Reprod Immunol 1989; 21: 151-154.

65. Morre D J, Kim C, Paulik M, Morre D M and Faulk W P. Is the drug-response NADH-oxidase of the cancer cell plasma membrane a molecular target for doxorubicin? Bioenerg Biomembr 1997; 29: 269-280.

66. Sun I L, Sun E E, Crane F L, Morre D J and Faulk W P. Inhibition of transplasma membrane electron transport by transferrin-doxorubicin conjugates. Biochim Biophy Acta 1992; 1105: 84-88.

67. Faulk W P, Barabas K, Sun I L and Crane F L. Transferrin-doxorubicin conjugates which inhibit tumor cell proliferation without interaction with DNA inhibit plasma membrane oxidoreductase and proton release in K562 cells. Biochem Int 1991; 25: 815-822.

68. Hileti D, Panayiotidis P and Hoffbrand V. Iron chelators induce apoptosis in proliferating cells. Brit J Haematol 1995; 89: 181-187.

69. Leardi A, Caraglia M, Selleri C, Pepe S, Pizzi C, Notaro R, Fabbrocini A, De Lorenzo S, Musico M, Abbruzzese A, Bianco A and Tagliaferri P. Desferioxamine increases iron depletion and apoptosis induced by ara-C of human myeloid leukemic cells. Brit J Haematol 1998; 102: 746-752.

70. Barabas K, Miller S J and Faulk W P. Regulation of transferrin receptor mRNA stability in drug-sensitive and drug-resistant cancer cells. To be submitted for publication, 2001.

71. Hentze M W and Kuhn L C. Molecular control of vertebrate iron-metabolism: mRNA-based regulatory circuits operated by iron, nitric oxide and oxidative stress. Proc Natl Acad Sci USA 1996; 93: 8175-8182.

72. Pantapoulos K and Hentze M W. Rapid responses to oxidative stress mediated by iron regulatory protein. EMBO J 1995; 14: 2917-1924.

73. Richardson D R, Naumannova V, Nagy E and Ponka P. The effect of redox-related species of nitrogen monoxide on transferrin and iron uptake and cellular proliferation of erythroleukemia (K562) cells. Blood 1995; 86: 3211-3219.

74. Laske D W, Ilercil O, Akbasak A, Youle R J and Oldfield E H. Efficacy of direct intratumoral therapy with targeted protein toxins for solid human gliomas in nude mice. J Neurosurg 1994; 80: 520-526.

75. Singh M, Atwal H and Micetich R. Transferrin directed delivery of doxorubicin to human cells. Anticancer Res 1998; 18(3A): 1423-1427.

76. Sato Y, Yamauchi N, Takahashi M, Sasaki K, Fukaura J, Neda H, Fujii S, Hirayma M, Itoh Y, Koshita Y, Kogawa K, Kato J, Sakamaki S and Niitsu Y. In vivo gene delivery to tumor cells by transferrin-streptavidin-DNA conjugate. FASEB Journal 2000; 14: 2108-2118.

77. Faulk W P, Taylor C G, Yeh G and McIntyre J A Preliminary clinical study of transferrin-doxorubicin conjugate for drug delivery to acute leukemia patients. Mol Biother 1990; 2: 57-60.

78. Laske D W, Morrison P F, Lieberman D M, Carthesy M E, Reynolds J C, Stewart-Henney PA, Koong S S, Cummins A, Paik C H and Oldfield E H. Chronic interstitial infusion of protein to primate brain: determination of drug distribution and clearance with single-photon emission computerized tomography imaging. J Neurosurg 1997; 87: 586-594.

79. Marbeuf-Gueye C, Ettori D, Priebe W, Kozlowski H and Garnier-Suillerot A Correlation between the kinetics of anthracycline uptake and the resistance factor in cancer cells expressing the multidrug resistance protein or the P-glycoprotein. Biochem Biophy Acta 1999; 1450: 374-384.

80. Klausner R D, vanReuswoude J, Ashwell G, Kempf C, Schechter A N, Dean A and Bridges K Receptor-mediated endocytosis of transferrin in K562 cells. J Biol Chem 1983; 258: 4715-4724.

81. Berczi A, Ruthner M, Szuts V, Fritzer M, Schweinzer E and Goldenberg H. Influence of conjugation of doxorubicin to transferrin on the iron uptake by K562 cells via receptor-mediated endocytosis. Euro J Biochem 1993; 213: 427-436.

82. Fritzer M, Barabas K, Szuts V, Berczi A, Szekeres T, Faulk W P and Goldenberg H. Cytotoxicity of a transferrin-doxorubicin conjugate to anthracylcine resistant cells. Int J Cancer 1992; 52: 619-623.

83. Hatano T, Ohkawa K and Matsuda M. Cytotoxic effect of the protein-doxorubicin conjugates on the multidrug-resistant human myelogenous leukemia cell line, K562, in vitro. Tumor Biology 1993; 14: 288-294.

84. Lemieux P and Page M. Sensitivity of multidrug-resistant MCF-7 cells to a transferrin-doxorubicin conjugate. Anticancer Res 1994; 14(2A): 397-403.

85. Fritzer M, Szekeres T, Szuts V, Jraayam H N and Goldenberg H. Cytotoxic effects of a doxorubicin-transferrin conjugate in multidrug-resistant KB cells. Biochem Pharm 1996; 51: 489-493.

86. Wang F, Jiang X, Yang D C, Elliot R L and Head J F. Doxorubicin-gallium-transferrin conjugate overcomes multidrug resistance: evidence for drug accumulation in the nucleus of drug resistant MCF-7/ADR cells. Anticancer Res 2000; 20: 799-808.

87. Soma C E, Dubernet C and Barratt G. Ability of doxorubicin-loaded nanoparticles to overcome multidrug resistance of tumor cells after their capture by macrophages. Pharm Res 1999; 16: 1710-1716.

88. Mazel M, Clair P, Rousselle C, Vidal P, Scherrmann J-M, Mathieu D and Temsamani J. Doxorubicin-peptide conjugates overcome multidrug resistance. Anti-Cancer Drugs 2001; 12: 107-116.

89. Barabas K, Sizensky J A and Faulk W P. Transferrin conjugates of doxorubicin are cytotoxic without intercalating nuclear DNA J Biol Chem 1992; 267: 9437-9442.

90. Barabas K and Faulk W P. Transferrin receptors associate with drug resistance in cancer cells. Biochem Biophys Res Com 1993; 197: 702-708.

1. A method for making a transferrin-doxorubicin conjugate having a predetermined ratio of doxorubicin to transferrin comprising the steps of:
a) adding the doxorubicin to a glutaraldehyde linker material in a manner that effectively results in the addition of each doxorubicin molecule to one glutaraldehyde molecule; and
b) adding the doxorubicin/glutaraldehyde combination to transferrin in a manner that results in a conjugate predetermined ratio of transferrin to doxorubicin;
wherein said transferrin-doxorubicin conjugate is substantially free of diners, trimers and aggregates.

2. The method according to claim 1, further comprising scavenging any excess glutaraldehyde linker.

3. A method for making a conjugate having a predetermined drug: protein ratio, comprising
a) adding a solution of a drug dropwise to a molar excess of a linker molecule solution to link each drug molecule to one linker molecule in a drug/linker combination; and
b) adding the drug/linker combination to a protein targeting agent to produce a conjugate having the predetermined drug: protein ratio,
wherein said conjugate is substantially free of dimers, trimers and aggregates.

4. The method according to claim 3, further comprising scavenging any excess linker.

5. The method according to claim 3, wherein said linker is glutaraldehyde.

6. The method according to claim 3, wherein said drug is selected from the group consisting of doxorubicin, methotrexate, vincristin, doxorubicin, daunomycin, 6-mercaptopurine, cytosine arabinoside, and cyclophosphamide.

7. The method according to claim 3, wherein said protein is selected from the group consisting of transferrin, ceruloplasmin, vitamins, vitamin binding proteins, hormones, cytokines, low density lipoproteins, and growth factors.

8. A substantially homogeneous material comprising a protein conjugated with a bioaffecting molecule in a predetermined ratio of bio-affecting molecules to protein molecules, wherein said protein is attracted to receptors on target cells.

9. The material according to claim 8, wherein the protein is transferrin.

10. The material according to claim 8, wherein the bio-affecting molecule is doxorubicin.

11. The material according to claim 8, wherein the predetermined ratio of bio-affecting molecules to protein molecules is between 0.1:1.0 and 4:1.0.

12. The material according to claim 8, wherein said bio-affecting molecule is selected from the group consisting of anti-cancer drugs, photosensitizers, heat sensitizers, apoptosis inducing materials, anti-viral agents, anti-protozoan agents and imaging aids.

13. The method according to claim 12, wherein said imaging aid is a radioactive isotope of iodine, gallium, indium, and yttrium

14. The method according to claim 13, wherein said radioactive isotope of iodine is selected from the group consisting of ¹²⁵I., ¹³¹I, ¹¹¹In, ⁹⁰Y, and ⁶⁷Ga.

15. A method for selectively treating target cells comprising contacting the cells with the material according to claim 8.

16. A homogeneous monomeric material suitable for bio-affecting target cells, said material consisting essentially of a monomeric conjugate, said monomeric conjugate comprising a protein that is attachable to receptors found in abundance on the target cells and a bio-affecting active molecule in a predetermined ratio of the bio-affecting active molecule to the protein, wherein said material is substantially free of dimers, trimers and aggregates.

17. The material according to claim 16, wherein the ratio of the bio-affecting active molecule to the protein is 0.2:1.0 to 8.0:1.0.

18. The material according to claim 14, wherein the ratio is 0.1:1.0 to 4.0:1.0.

19. A reagent kit for the treatment of tumors, comprising iron-bearing transferrin, and a homogeneous conjugate with a predetermined and consistent number of antitumor agent molecules per molecule of transferrin.

20. A reagent kit for determining the susceptibility of tumor cells to anti-tumor agents, comprising two or more homogeneous conjugates with a predetermined and consistent number of antitumor agent molecules per molecule of transferrin, wherein said homogeneous conjugates have different antitumor agents.

21. A method of treating a subject having a tumor susceptible to anti-tumor therapy, said method comprising administering to a patient an anti-tumor effective amount of the substantially homogeneous material of claim 8.

22. The method according to claim 21, wherein the protein is transferrin.

23. The method according to claim 21, wherein the bio-affecting molecule is doxorubicin.

24. The method according to claim 21, further comprising imaging said tumor.

25. The method according to claim 24, wherein said tumor is imaged using the substantially homogeneous material of claim 8, wherein the bio-affecting material is selected from the group consisting of isotopes, fluorescent molecules and radio opaqing materials.

26. The method according to claim 21, wherein the predetermined ratio of bio-affecting molecules to protein molecules is between 0.1:1.0 and 4.0:1.0.

27. A method for treating drug resistant cells, comprising administering the substantially homogeneous material of claim 8 to said cells.

28. A method for the targeted inhibition of a plasma membrane redox enzyme in a tumor cell, comprising administering the substantially homogeneous material of claim 8 to said tumor cell to inhibit said enzyme.

29. The method according to claim 28, wherein said enzyme is NADH-oxidase or NADH-reductase.

30. The method according to claim 28, wherein said protein is transferrin.

31. The method according to claim 28, wherein said bioaffecting molecule is doxorubicin.

32. The method according to claim 28, wherein said tumor cell is in a patient and said substantially homogeneous material is administered to said patient.

33. A method for inducing apoptosis in drug resistant tumor cells by destabilizing transferrin receptor mRNA, comprising administering the substantially homogeneous material of claim 8 to said tumor cell.

34. The method according to claim 33, wherein said protein is transferrin.

35. The method according to claim 33, wherein said bioaffecting molecule is doxorubicin.

36. The method according to claim 33, wherein said tumor cell is in a patient and said substantially homogeneous material is administered to said patient.

37. A method for destabilizing transferrin receptor mRNA in drug resistant cancer cells, comprising administering the substantially homogeneous material according to claim 8 to said cancer cells to trap iron and thereby destabilize said transferrin receptor mRNA.

38. The method according to claim 37, wherein said protein is transferrin.

39. The method according to claim 37, wherein said bioaffecting molecule is deferoxamine.

40. The method according to claim 37, wherein said tumor cell is in a patient and said substantially homogeneous material is administered to said patient.

41. A method for making a conjugate having a predetermined drug: protein ratio, comprising
a) adding a solution of a linker molecule drop-wise to solution of a cryopreservative to produce a first intermediate product,
b) adding a solution of a drug dropwise to a molar excess of said first intermediate product to link each drug molecule to one linker molecule in a drug/linker combination to produce a second intermediate product; and
c) adding the second intermediate product to a protein targeting agent to produce a third intermediate product having the predetermined drug:
protein ratio,
d) adding a linker scavenging agent to said third intermediate product to produce a fourth intermediate product, and
e) filtering said fourth intermediate product to obtain a conjugate having a predetermined drug: protein ratio,
wherein said conjugate is substantially free of dimers, trimers and aggregates.
